(19) United States
(12) Patent Application Publication
(10) Pub. No.: US 2019/0202856 A1
(43) Pub. Date: Jul. 4, 2019

(54) ENGINEERED CRISPR PROTEINS FOR COVALENT TAGGING NUCLEIC ACIDS

(71) Applicant: SIGMA-ALDRICH CO. LLC, St. Louis, MO (US)

(72) Inventors: Gregory D. Davis, St. Louis, MO (US); Daniel Taglicht, St. Louis, MO (US); Fuqiang Chen, St. Louis, MO (US)

(21) Appl. No.: 16/221,066

(22) Filed: Dec. 14, 2018

Related U.S. Application Data
(60) Provisional application No. 62/611,727, filed on Dec. 29, 2017.

(51) Int. Cl. C07H 21/04; C12N 9/22; C12N 15/62; C12N 15/10

(52) U.S. Cl. CPC C07H 21/04; C12N 9/22; C12N 2310/3517; C12N 15/102; C12N 2310/20; C12N 15/62

(57) ABSTRACT

CRISPR proteins engineered to form covalent bonds with 5′ phosphates in target nucleic acids and methods of using CRISPR systems comprising said engineered CRISPR proteins to covalently tag nucleic acids.

### CROSS-REFERENCE TO RELATED APPLICATIONS

This application claims priority under 35 U.S.C. 119(e) to U.S. Provisional Application Ser. No. 62/611,727, filed Dec. 29, 2017, the disclosure of each is hereby incorporated by reference in its entirety

### SEQUENCE LISTING

The instant application contains a Sequence Listing which has been submitted electronically in ASCII format and is hereby incorporated by reference in its entirety. Said ASCII copy, created on Dec. 10, 2018, is named P17_326_SL.TXT and is 8,695 bytes in size.

### FIELD

The present disclosure relates to CRISPR proteins engineered to form covalent bonds with 5′ phosphates in target nucleic acid and methods of using CRISPR systems comprising said engineered CRISPR proteins to covalently tag nucleic acids.

### BACKGROUND

RNA-guided clustered regularly interspersed short palindromic repeats (CRISPR) systems have been widely adopted into live cell genome editing applications. As programmable DNA binding complexes, CRISPR system can also be used to label and/or isolate nucleic acids in a sequence specific manner (Deng et al., Proc. Natl. Acad. Sci. USA, 2015, 112(38):11870-11875). However, chromatin immunoprecipitation (ChIP) studies have shown that the number of sites bound by catalytically inactive Cas9 greatly exceeds those which undergo a Cas9-induced double strand break (Deng et al. 2015). Thus, for CRISPR systems to be useful for nucleic acid recognition and isolation, improvements in binding specificity are critical.

### SUMMARY

Among the various aspects of the present disclosure provides an engineered CRISPR protein. The engineered CRISPR protein comprises at least one modification such that the CRISPR protein is capable of forming a covalent bond with a 5′-phosphate within a target nucleic acid sequence.

Another aspect of the present disclosure encompasses a method for detecting a nucleic acid. The method comprises (a) contacting the nucleic acid with a CRISPR system comprising (i) a CRISPR protein engineered to comprise at least one modification such that it is capable of forming a covalent linkage with a cleaved nucleic acid and (ii) a guide RNA, wherein the guide RNA guides the CRISPR protein to a target sequence in the nucleic acid and the CRISPR protein forms a covalent bond with a 5′-phosphate within the target sequence to form a CRISPR protein-nucleic acid complex; and (b) detecting the CRISPR protein-nucleic acid complex.

Other aspects and features of the disclosure are detailed bellow.

### DETAILED DESCRIPTION

The present disclosure provides engineered CRISPR proteins that are capable of covalently attaching to a 5′ phosphate in target nucleic acid. Since CRISPR double-stranded break (DSB) activity is tightly regulated by changes in Cas9 or Cpf1 protein tertiary structure (Chen et al., Nature, 2017, 550:407-410; Gao et al., Cell Research, 2016, 26(8):901-913), it was reasoned that placing a possible covalent attachment site under the same structural regulation as DSB activity could result in a highly specific and stable covalent DNA attachment. In general, the engineered CRISPR protein comprises at least one modification (e.g., a tyrosine or serine substitution) that is in spatial proximity such that the CRISPR protein can form a covalent bond with a 5′-phosphate present within the target nucleic acid. Also provided herein are methods for labeling and detecting nucleic acids of interest, wherein the method comprises contacting the nucleic acid of interest with a CRISPR system comprising the engineered CRISPR protein and a guide RNA such that the engineered CRISPR protein forms a covalent attachment with a 5′-phosphate in the nucleic acid of interest, wherein the covalent attachment is insensitive to proteases and denaturing conditions (e.g., acid solution, alkaline solutions, phenol, 8 M urea, SDS, heat, and the like).

### (I) Engineered CRISPR Proteins

One aspect of the present disclosure provides a CRISPR protein engineered to comprise at least one modification such that the CRISPR protein is capable of forming a covalent bond with a 5′-phosphate within a target nucleic acid sequence. Such engineered CRISPR proteins, therefore, are capable of covalently tagging nucleic acids.

The modification of the engineered CRISPR protein can be a substitution of one or more amino acids, an insertion of one or more amino acids, a deletion of one or more amino acids, an insertion of a domain from a protein known to covalently bind nucleic acids, a replacement of a CRISPR protein domain with a domain from a protein known to covalently bind nucleic acids, or a combination thereof.

(a) CRISPR Proteins

The CRISPR protein that is engineered to covalently bind a nucleic acid can be derived from any naturally-occurring, modified, or genetically-engineered CRISPR protein.

In certain embodiments, the engineered CRISPR protein can be derived from a type I (i.e., IA, IB, IC, ID, IE, or IF), type II (i.e., IIA, IIB, or IIC), type III (i.e., IIIA or IIIB), type V, or type VI CRISPR system, which are present in various bacteria and archaea. For example, the CRISPR protein can be from Streptococcus sp. (e.g., S. pyogenes, S. thermophilus, S. pasteurianus), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lachnospiraceae sp., Lactobacillus sp., Leptotrichia sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., Thermosipho sp., or Verrucomicrobia sp. In certain embodiments the CRISPR protein can be derived from thermophilic species such as Geobacillus stearothermophilus.

In other embodiments the CRISPR protein can be derived from Acidothermus cellulolyticus, Alicyclobacillus hesperidum, Francisella tularensis subsp. novicida, Parasutterella excrementihominis, Wolinella succinogenes, Mycoplasma canis, Ralstonia syzygii, Bifidobacterium bombi, Oenococcus kitaharae, Nitratifractor salsuginis, Bacillus smithii, Akkermansia muciniphila, Corynebacterium diptheriae, Lactobacillus rhamnosus, Akkermansia glycaniphila, Mycoplasma gallisepticum, Parvibaculum lavamentivorans.

Non-limiting examples of suitable CRISPR proteins include Cas proteins (e.g., Cas9, Cas1, Cas2, Cas3, Cas13a and the like), Cpf proteins, C2c proteins (e.g., C2c1, C2c2, C2c3), Cmr proteins, Csa proteins, Csb proteins, Csc proteins, Cse proteins, Csf proteins, Csm proteins, Csn proteins, Csx proteins, Csy proteins, Csz proteins, and derivatives or variants thereof.

In some embodiments, the engineered CRISPR protein can be derived from a type II CRISPR/Cas9 system. In other embodiments, the CRISPR protein can be derived from a type V CRISPR/Cpf1 system. In further embodiments, the CRISPR protein can be derived from a CRISPR/CasX system or a CRISPR/CasY system (Burstein et al., Nature, 2017, 542(7640):237-241). In additional embodiments, the CRISPR protein can be derived from a type VI CRISPR/Cas13a (formerly C2c2) system.

In various embodiments, the engineered CRISPR protein can be derived from Streptococcus pyogenes Cas9 (SpCas9), Streptococcus thermophilus Cas9 (StCas9), or Streptococcus pasteurianus (SpaCas9). In other embodiments, the CRISPR protein can be derived from Campylobacter jejuni Cas9 (CjCas9). In alternate embodiments, the CRISPR protein can be derived from Francisella novicida Cas9 (FnCas9). In still other embodiments, the CRISPR protein can be derived from Neisseria cinerea Cas9 (NcCas9). In further embodiments, the CRISPR protein can be derived from Francisella novicida Cpf1 (FnCpf1), Acidaminococcus sp. Cpf1 (AsCpf1), or Lachnospiraceae bacterium ND2006 Cpf1 (LbCpf1). In still other embodiments, the CRISPR protein can be derived from Leptotrichia wadei Cas13a (LwaCas13a) or Leptotrichia shahii Cas13a (LshCas13a).

In general, CRISPR proteins comprise at least one nuclease domain having endonuclease activity. For example, a Cas9 protein comprises a RuvC nuclease domain and an HNH nuclease domain; a Cpf1 protein comprises a RuvC domain and another nuclease domain (NUC); and a Cas13a comprises two HNEPN domains. CRISPR proteins also comprise RNA recognition and/or RNA binding domain, which interacts with the guide RNA, a recognition domain that interacts with the RNA/DNA heteroduplex, and regions that recognize and interact with a protospacer adjacent motif (PAM) sequence in the target nucleic acid.

In certain embodiments, the engineered CRISPR protein can be derived from a wild type or naturally-occurring protein. In other embodiments, the engineered CRISPR protein can be derived from a CRISPR protein engineered to have improved specificity, reduced non-specific DNA contacts, altered PAM specificity, decreased off-target effects, increased stability, and the like. For example, the CRISPR protein can be high-fidelity variant of Cas9 protein, or a hyper-accurate variant of Cas9 protein.

In yet other embodiments, the engineered CRISPR protein can be derived from a CRISPR protein modified to cleave only one strand of DNA. A CRISPR nuclease can be converted to a CRISPR nickase by one or more alterations in the protein, wherein the alteration inactivates one of the nuclease domains and the nickase cleaves only one strand of a double-stranded sequence. The modification can be a substitution of one or more amino acids, an insertion of one or more amino acids, a deletion of one or more amino acids, or a combination thereof. For example, a Cas9 nickase can comprise one or more alterations in one of the nuclease domains (e.g., the RuvC domain or the HNH domain). In some embodiments, the one or more alterations can be D10A, D8A, E762A, and/or D986A substitution in the RuvC domain (such that the RuvC domain is catalytically inactive) or the one or more alterations can be H840A, H559A, N854A, N856A, and/or N863A substitution in the HNH domain (such that the HNH domain is catalytically inactive). In other embodiments, a Cpf1 nickase can comprise one or more alterations in one of the nuclease domains (e.g., RuvC or NUC).

(b) Modification

The engineered CRISPR protein disclosed herein comprises at least one modification such that the CRISPR protein is capable of forming a covalent bond with a 5′-phosphate within a target nucleic acid. The at least one modification can be a substitution of one or more amino acids, an insertion of one or more amino acids, a deletion of one or more amino acids, an insertion of a domain from a protein known to covalently bind nucleic acids, a replacement of a CRISPR protein domain with a domain from a protein known to covalently bind nucleic acids, or combinations thereof.

In general, the at least one modification is located within a domain that undergoes structural movement to arrive in closer spatial proximity to the DNA target, wherein such movement is dependent upon sufficient base pairing between the guide RNA and the target DNA, and such movement is extensive enough for the at least one modification to chemically act upon the DNA target and result in a covalent bond. In some embodiments, the at least one modification is located within a nuclease domain of the CRISPR protein. For example, the at least one modification can be located within the HNH nuclease domain of a Cas9 protein, the at least one modification can be located within the RuvC nuclease domain of a Cas9 protein, the at least one modification can be located within the NUC nuclease domain of a Cpf1 protein, or the at least one modification can be located within the RuvC nuclease domain of a Cpf1 protein.

In some embodiments, the engineered CRISPR protein can comprise one or more amino acid substitutions. For example, amino acid residues at specific locations in a wild type CRISPR protein can be changed to tyrosine, serine, aspartic acid, or glutamic acid residues, such that, upon heteroduplex formation between the guide RNA and the target nucleic acid, the CRISPR protein undergoes structural changes that facilitate formation of a covalently linkage between the tyrosine, serine, aspartic acid, or glutamic acid residue and a 5′ phosphate in the target nucleic acid. In certain embodiments, the engineered CRISPR protein can comprise strategically-located tyrosine and/or serine residues in the mobile HNH nuclease domain of a Cas9 protein or the mobile NUC domain of a Cpf1 protein such that tyrosyl-phosphodiester and/or seryl-phosphodiester bond(s) are formed between the CRISPR protein and 5′ phosphate(s) of the target nucleic acid.

In alternate embodiments, the engineered CRISPR protein can be modified to include non-canonical amino acids (ncAA) that enable or enhance the chemical reaction scheme required for covalent and stable attachment of a protein to a DNA, RNA, or other nucleic acid. Such ncAAs have been previously shown to function in both prokaryotic and eukaryotic systems via engineering of the genetic code, associated tRNA synthetases, and other proteins and nucleic acids which support creation and integration of ncAAs into recombinant proteins (Young et al., J. Biol. Chem., 2010, 285(15):11039-11044).

In further embodiments, the HNH, NUC, or other domain of the CRISPR protein can be fused to or entirely replaced with a domain or fragment thereof of a protein known to covalently bind to DNA or other nucleic acids. Suitable proteins with domains capable of covalent DNA binding include: (1) prokaryotic and eukaryotic topoisomerases, e.g., the Col EI relaxation complex, (2) tyrosine or serine recombinases, e.g., Cre, Flp, ϕC31, XerC, XerD, and the like, (3) rolling circle plasmid replication proteins such as the RepA protein of plasmid pC194 or the RepC protein of pT181, (4) the Rep protein of the rolling circle phage ϕX174, (5) the p5 Rep protein (Rep68) of adeno-associated virus (AAV), the VPg proteins of EMC, poliovirus, picornavirus, or comovirus, (6) HUH class endonucleases including PCV2 from porcine circovirus 2, DCV from duck circovirus, FBNYV from favabean necrosis yellow virus, RepB from Streptococcus agalactiae, RepBm from Fructobacillus tropaeoli, TarI from E. coli, mob from E. coli, NEST from Staphylococcus aureus, (7) wild type and engineered derivatives of O⁶-alkylguanine-DNA alkyltransferase (AGT) used in covalent labeling, wild type and engineered derivatives of acyl carrier protein (ACP), and (8) in scenarios where the target nucleic acid is chemically modified, certain protein classes become applicable for covalent entrapment, for example 5-fluoro-C can form covalent adducts with DNA (cytosine-5)-methyltransferases (including DNMT1, DNMT3A, and DNMT3B).

(c) Optional Domains

In some embodiments, the engineered CRISPR protein can further comprise at least one nuclear localization signal (NLS). Non-limiting examples of nuclear localization signals include PKKKRKV (SEQ ID NO:1), PKKKRRV (SEQ ID NO:2), KRPAATKKAGQAKKKK (SEQ ID NO:3), YGRKKRRQRRR (SEQ ID NO:4), RKKRRQRRR (SEQ ID NO:5), PAAKRVKLD (SEQ ID NO:6), RQRRNELKRSP (SEQ ID NO:7), VSRKRPRP (SEQ ID NO:8), PPKKARED (SEQ ID NO:9), PQPKKKPL (SEQ ID NO:10), SALIKKKKKMAP (SEQ ID NO:11), PKQKKRK (SEQ ID NO:12), RKLKKKIKKL (SEQ ID NO:13), REKKKFLKRR (SEQ ID NO:14), KRKGDEVDGVDEVAKKKSKK (SEQ ID NO:15), RKCLQAGMNLEARKTKK (SEQ ID NO:16), NQSSNFGPMKGGNFGGRSSGPYGGGGQYFAKPRNQGGY (SEQ ID NO:17), and RMRIZFKNKGKDTAELRRRRVEVSVELRKAKKDEQILKRRNV (SEQ ID NO:18).

In further embodiments, the engineered CRISPR protein can further comprise at least one cell-penetrating domain. Examples of suitable cell-penetrating domains include, without limit, GRKKRRQRRRPPQPKKKRKV (SEQ ID NO:19), PLSSIFSRIGDPPKKKRKV (SEQ ID NO:20), GALFLGWLGAAGSTMGAPKKKRKV (SEQ ID NO:21), GALFLGFLGAAGSTMGAWSQPKKKRKV (SEQ ID NO:22), KETWWETWWTEWSQPKKKRKV (SEQ ID NO:23), YARAAARQARA (SEQ ID NO:24), THRLPRRRRRR (SEQ ID NO:25), GGRRARRRRRR (SEQ ID NO:26), RRQRRTSKLMKR (SEQ ID NO:27), GWTLNSAGYLLGKINLKALAALAKKIL (SEQ ID NO:28), KALAWEAKLAKALAKALAKHLAKALAKALKCEA (SEQ ID NO:29), and RQIKIWFQNRRMKWKK (SEQ ID NO:30).

In yet additional embodiments, the engineered CRISPR protein can further comprise at least one marker domain. Marker domains include fluorescent proteins and purification or epitope tags. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreen1, and so forth), yellow fluorescent proteins (e.g., YFP, EYFP, Citrine, Venus, YPet, PhiYFP, ZsYellow1, and the like), blue fluorescent proteins (e.g., BFP, EBFP, EBFP2, Azurite, mKalama1, GFPuv, Sapphire, T-sapphire, and so forth), cyan fluorescent proteins (e.g., ECFP, Cerulean, CyPet, AmCyan1, Midoriishi-Cyan, and the like), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred, and so forth), and orange fluorescent proteins (e.g., mOrange, mKO, Kusabira-Orange, Monomeric Kusabira-Orange, mTangerine, tdTomato, and the like). Non-limiting examples of suitable purification or epitope tags include biotin, glutathione-S-transferase (GST), chitin binding protein (CBP), maltose binding protein, thioredoxin (TRX), poly(NANP), tandem affinity purification (TAP) tag, myc, His, poly-His, FLAG, HA, AcV5, AU1, AUS, E, ECS, E2, nus, Softag 1, Softag 3, Strep, SBP, Glu-Glu, HSV, KT3, S, S1, T7, V5, VSV-G, biotin carboxyl carrier protein (BCCP), and calmodulin.

In further embodiments, the engineered CRISPR protein can further comprise at least one detectable label. The detectable label can be a detection tag (e.g., biotin, digoxigenin, or dinitrophenyl), a fluorescent dye (e.g., fluorescein or derivatives thereof (e.g., FAM, HEX, TET, TRITC), rhodamine or derivatives thereof (e.g., ROX), Texas Red, cyanine dyes, Alexa dyes, diethylaminocoumarin, and the like), a fluorescent quencher (e.g., black hole quenchers (BHQs) such as BHQ-0, BHQ-1, BHQ-2, BHQ-3, deep dark quencher such as DDQ-I or DDQ-II, Iowa black quenchers, QSY quenchers, and so forth), or combinations thereof (e.g., a FRET fluorophore and quencher pair). In other embodiments, the detectable label can be a peptide tag (such as the AviTag) which undergoes post-translational modification to create a detection tag such as the attachment of biotin via the E. coli biotin ligase (BirA), or wild type and engineered derivatives of O⁶-alkylguanine-DNA alkyltransferase (AGT), or wild type and engineered derivatives of acyl carrier protein (ACP). The label attached to the CRISPR protein can also be a nucleic acid which enables detection via hybridization, hybridization chain reaction, or replicative methods such as the polymerase chain reaction or rolling circle amplification.

### (II) CRISPR Systems Comprising Engineered CRISPR Proteins

Another aspect of the present disclosure comprises a CRISPR system comprising an engineered CRISPR protein and a guide RNA.

(a) Engineered CRISPR Proteins

Engineered CRISPR proteins are described above in section (I).

(b) Guide RNA

The guide RNA has complementarity to a target sequence in the nucleic acid of interest. The guide RNA interacts with the CRISPR protein and the target sequence (i.e., protospacer sequence) such that it guides the CRISPR protein to the target sequence at which site the CRISPR protein cleaves the target sequence. The target sequence has no sequence limitation except that the sequence is adjacent to a protospacer adjacent motif (PAM). For example, PAM sequences for Cas9 proteins include 5′-NGG, 5′-NGGNG, 5′-NNAGAAW, and 5′-ACAY, and PAM sequences for Cpf1 include 5′-TTN (wherein N is defined as any nucleotide, W is defined as either A or T, and Y is defined an either C or T).

A guide RNA can comprise three regions: a first region at the 5′ end that has complementarity to the target sequence in the nucleic acid of interest, a second region that is internal and forms a stem loop structure, and a third region at the 3′ end that can remain single-stranded. The second and third regions form a secondary structure that interacts with the CRISPR protein. The first region of each guide RNA is different (i.e., is sequence specific). The second and third regions can be the same in guide RNAs that complex with a particular CRISPR protein.

The first region of the guide RNA has complementarity to the target sequence such that the first region of the guide RNA can base pair with the target sequence. For example, the first region of a SpCas9 guide RNA can comprise GN₁₇₋₂₀GG. In general, the complementarity between the first region (i.e., crRNA) of the guide RNA and the target sequence is at least 80%, at least 85%, at least 90%, at least 95%, or more. In various embodiments, the first region of the guide RNA can comprise from about 10 nucleotides to more than about 25 nucleotides. For example, the region of base pairing between the first region of the guide RNA and the target site in the chromosomal DNA sequence can be about 10, 11, 12, 13, 14, 15, 16, 17, 18, 19, 20, 22, 23, 24, 25, or more than 25 nucleotides in length. In an exemplary embodiment, the first region of the guide RNA is about 19, 20, or 21 nucleotides in length.

The guide RNA also comprises a second region that forms a secondary structure. In some embodiments, the secondary structure comprises at least one stem (or hairpin) and loop. The length of each loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. The guide RNA also comprises a third region at the 3′ end that can remain single-stranded. Thus, the third region has no complementarity to any nucleic acid sequence in the cell of interest and has no complementarity to the rest of the guide RNA. The length of the third region can vary. In general, the third region is more than about 4 nucleotides in length. For example, the length of the third region can range from about 5 to about 60 nucleotides in length. In some embodiments, the length of the third region can be extended to comprise one or more additional stem-loop regions.

The combined length of the second and third regions (also called the universal or scaffold region) of the guide RNA can range from about 30 to about 120 nucleotides in length. In one aspect, the combined length of the second and third regions of the guide RNA range from about 70 to about 100 nucleotides in length.

In some embodiments, the guide RNA can be a single molecule comprising all three regions. In other embodiments, the guide RNA can comprise two separate molecules. The first RNA molecule (i.e., crRNA) can comprise the first region of the guide RNA and one half of the “stem” of the second region of the guide RNA. The second RNA molecule (i.e., tracrRNA) can comprise the other half of the “stem” of the second region of the guide RNA and the third region of the guide RNA. Thus, in this embodiment, the first and second RNA molecules each contain a sequence of nucleotides that are complementary to one another. For example, in one embodiment, crRNA and tracrRNA molecules each comprise a sequence (of about 6 to about 20 nucleotides) that base pairs with the other sequence to form a functional guide RNA. For example, the guide RNA of type II CRISPR/Cas systems can comprise crRNA and tracrRNA. In some aspects, the crRNA for a type II CRISPR/Cas system can be chemically synthesized and the tracrRNA type II CRISPR/Cas system can be enzymatically synthesized in vitro. In other embodiments, the guide RNA of type V CRISPR/Cpf1 systems can comprise only crRNA.

The guide RNA can comprise standard ribonucleotides, modified ribonucleotides (e.g., pseudouridine), ribonucleotide isomers, and/or ribonucleotide analogs. In some embodiments, the guide RNA can further comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles. Those skilled in the art are familiar with gRNA design and construction, e.g., gRNA design tools are available on the internet or from commercial sources.

The guide RNA can be synthesized chemically, synthesized enzymatically, or a combination thereof. For example, the guide RNA can be synthesized using standard phosphoramidite-based solid-phase synthesis methods. Alternatively, the guide RNA can be synthesized in vitro by operably linking DNA encoding the guide RNA to a promoter control sequence that is recognized by a phage RNA polymerase. Examples of suitable phage promoter sequences include T7, T3, SP6 promoter sequences, or variations thereof. In embodiments in which the guide RNA comprises two separate molecules (i.e., crRNA and tracrRNA), the crRNA can be chemically synthesized and the tracrRNA can be enzymatically synthesized. The nucleic acid encoding the guide RNA can be part of a plasmid vector, which can further comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. DNA encoding the guide RNA can be operably linked to a promoter control sequence that is recognized by RNA polymerase III (Pol III) for expression in eukaryotic cells.

### (III) Nucleic Acids Encoding the Engineered CRISPR Proteins

A further aspect of the present disclosure provides nucleic acids encoding the engineered CRISPR proteins described above in section (I) or the CRISPR systems described above in section (II). The CRISPR systems can be encoded by single nucleic acids or multiple nucleic acids. The nucleic acids can be DNA or RNA, linear or circular, single-stranded or double-stranded. The RNA or DNA can be codon optimized for efficient translation into protein in the eukaryotic cell of interest. Codon optimization programs are available as freeware or from commercial sources.

In some embodiments, the nucleic acid encoding the engineered CRISPR protein can be RNA. The RNA can be enzymatically synthesized in vitro. For this, DNA encoding the engineered CRISPR protein can be operably linked to a promoter sequence that is recognized by a phage RNA polymerase for in vitro RNA synthesis. For example, the promoter sequence can be a T7, T3, or SP6 promoter sequence or a variation of a T7, T3, or SP6 promoter sequence. The DNA encoding the engineered CRISPR protein can be part of a vector, as detailed below. In such embodiments, the in vitro-transcribed RNA can be purified, capped, and/or polyadenylated. In other embodiments, the RNA encoding the CRISPR protein can be part of a self-replicating RNA (Yoshioka et al., Cell Stem Cell, 2013, 13:246-254). The self-replicating RNA can be derived from a noninfectious, self-replicating Venezuelan equine encephalitis (VEE) virus RNA replicon, which is a positive-sense, single-stranded RNA that is capable of self-replicating for a limited number of cell divisions, and which can be modified to code proteins of interest (Yoshioka et al., Cell Stem Cell, 2013, 13:246-254).

In other embodiments, the nucleic acid encoding the engineered CRISPR protein or the CRISPR system can be DNA. The DNA coding sequence can be operably linked to at least one promoter control sequence for expression in the cell of interest. In certain embodiments, the DNA coding sequence can be operably linked to a promoter sequence for expression of the CRISPR protein or system in bacterial (e.g., E. coli) cells or eukaryotic (e.g., yeast, insect, or mammalian) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-8 promoter, Mb promoter, NphsI promoter, OG-2 promoter, SP-B promoter, SYN1 promoter, and WASP promoter. The promoter sequence can be wild type or it can be modified for more efficient or efficacious expression. In some embodiments, the DNA coding sequence also can be linked to a polyadenylation signal (e.g., SV40 polyA signal, bovine growth hormone (BGH) polyA signal, etc.) and/or at least one transcriptional termination sequence. In some situations, the CRISPR protein or system can be purified from the bacterial or eukaryotic cells.

In various embodiments, nucleic acid encoding the CRISPR protein or CRISPR system can be present in a vector. Suitable vectors include plasmid vectors, viral vectors, and self-replicating RNA (Yoshioka et al., Cell Stem Cell, 2013, 13:246-254). In some embodiments, the nucleic acid encoding the CRISPR protein or system can be present in a plasmid vector. Non-limiting examples of suitable plasmid vectors include pUC, pBR322, pET, pBluescript, and variants thereof. In other embodiments, the nucleic acid encoding the CRISPR protein or system can be part of a viral vector (e.g., lentiviral vectors, adeno-associated viral vectors, adenoviral vectors, and so forth). The plasmid or viral vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some embodiments, vectors comprising sequence encoding the CRISPR protein can further comprise sequence encoding at least one guide RNA. The sequence encoding the guide RNA generally is linked to at least one promoter control sequence for expression of the guide RNA in the eukaryotic cell of interest. For example, sequence encoding the guide RNA can be operably linked to a promoter sequence that is recognized by RNA polymerase III (Pol III). Examples of suitable Pol III promoters include, but are not limited to, mammalian U6, U3, H1, and 7SL RNA promoters. Additional information about vectors and use thereof can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

### (IV) Methods for Detecting Nucleic Acids

Another aspect of the present disclosure encompasses methods for detecting a nucleic acid of interest, wherein the methods comprise (a) contacting the nucleic acid of interest with a CRISPR system comprising (i) a CRISPR protein engineered to comprise at least one modification such that it is capable of forming a covalent linkage with a nucleic acid and (ii) a guide RNA, wherein the guide RNA guides the CRISPR protein to a target sequence in the nucleic acid and the CRISPR protein forms a covalent bond with a 5′-phosphate within the target sequence to form a CRISPR protein-nucleic acid complex, and (b) detecting the CRISPR protein-nucleic acid complex.

CRISPR systems comprising engineered CRISPR proteins that are capable of covalently tagging the 5′-end of cleaved nucleic acids are detailed above in section (II).

In embodiments in which the nucleic acid of interest is within a live cell, the contacting step comprises introducing into the cell the engineered CRISPR protein or a nucleic acid encoding the engineered CRISPR protein and the guide RNA or a nucleic acid encoding the guide RNA. Nucleic acids encoding the CRISPR protein and/or guide RNA are detailed above in section (III).

The appropriate molecules (i.e., protein, RNA, protein-RNA complex, and/or DNA) can be introduced into the live cell by a variety of means. In some embodiments, the cell can be transfected with the appropriate molecules. Suitable transfection methods include nucleofection (or electroporation), calcium phosphate-mediated transfection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001). In other embodiments, the appropriate molecules can be introduced into the cell by microinjection. For example, the molecules can be injected into the cytoplasm or nuclei of the live cells of interest. The amount of each molecule introduced into the cell can vary, but those skilled in the art are familiar with means for determining the appropriate amount.

(a) Nucleic Acids for Detection

Nucleic acids detected by the methods disclosed herein can be DNA or RNA. Those of skill in the art understand that the choice of CRISPR protein included in the CRISPR system will determine the type of nucleic acid that can be detected. For example, CRISPR proteins such as Cas9 and Cpf1 target DNA, whereas Cs13a targets RNA.

In some embodiments, the nucleic acid can be within a cell. For example, the nucleic acid can be chromosomal DNA, extrachromosomal DNA, nuclear DNA, cytoplasmic DNA, mitochondrial DNA, plastid DNA, nuclear RNA, or cytoplasmic RNA. In some embodiments, the cell can be in vivo (i.e., disposed in an organism). In other embodiments, the cell can be ex vivo. For example, the cell can be within an organ or part of an organ removed from an organism (e.g., a tissue biopsy). In still other embodiments, the cell can be a cultured cell or a cell line cell.

In general, the cell is a eukaryotic cell. For example, the cell can be a human cell, a non-human mammalian cell, a non-mammalian vertebrate cell, an invertebrate cell, an insect cell, a plant cell, a yeast cell, or a single cell eukaryotic organism. In some embodiments, the cell can also be a one cell embryo. In still other embodiments, the cell can be a stem cell such as embryonic stem cells, ES-like stem cells, fetal stem cells, adult stem cells, and the like. In other embodiments, the cell can be a diseased or cancerous cell. Non-limiting examples of suitable cell line cells include human cells lines (e.g., human embryonic kidney cells (HEK), human cervical carcinoma cells (HELA), human lung cells, human liver cells, human osteosarcoma cells, etc.), mouse cell lines (e.g., mouse myeloma NS0 cells, mouse embryonic fibroblast 3T3 cells, mouse B lymphoma A20 cells, mouse melanoma B16 cells, and so forth), Chinese hamster ovary (CHO) cells, baby hamster kidney (BHK) cells, monkey kidney cell lines, rat cell lines, canine cell lines, and so forth. An extensive list of mammalian cell lines may be found in the American Type Culture Collection catalog (ATCC, Manassas, Va.).

In certain embodiments, the cell can be a live cell. In other embodiments, the cell can be a frozen cell. In further embodiments, the cell can be a fixed cell. Cells can be fixed by contact with a fixative. Examples of suitable fixatives include acetone, acetic acid, ethanol, formaldehyde (or formalin, a 37% aqueous solution of formaldehyde), glutaraldehyde, iodoform, lactic acid, methanol, paraformaldehyde, picric acid, and combinations thereof. In some embodiments, the fixed cell can be contacted with a denaturing solution to convert double-stranded nucleic acids into single-stranded nucleic acids. The denaturing solution can be acidic or it can be alkaline. In further embodiments, the fixed cell can be embedded in a tissue embedding medium. Suitable tissue embedding media include paraffin, paraffin-based resins, epoxy resins, methacrylate resins, polyester waxes, and so forth. The embedded cells (or frozen cells) can be sectioned into thin slices (e.g., from about 0.5 micron to about 10 microns) prior to contact with the CRISPR system. In some embodiments, the cell can be within a formalin-fixed paraffin-embedded (FFPE) sample.

In alternate embodiments, the nucleic acid can be in vitro (i.e., in a test-tube or a cell-free mixture). The nucleic acid to be detected can be isolated from a cell, synthesized chemically in vitro, or chemically synthesized.

(b) Contact with the CRISPR System

The method comprises contacting the nucleic acid of interest with a CRISPR system comprising (i) an engineered CRISPR protein as disclosed herein and (ii) a guide RNA. In embodiments in which the nucleic acid is disposed in a live cell, the contacting step can comprise introducing into the cell (i) a complex comprising the CRISPR protein and the guide RNA, (ii) the CRISPR protein and a nucleic acid encoding the guide RNA, or (iii) a nucleic acid encoding the CRISPR protein and a nucleic acid encoding the guide RNA, and the live cell is cultivated at a temperature suitable for the cell. In embodiments in which the nucleic acid is disposed in a fixed cell, the contacting step can comprise contact with a complex comprising the CRISPR protein and the guide RNA, wherein the contacting can be conducted at a temperature ranging from about 40° C. to about 90° C. For example, access to formalin fixed DNA may be enhanced by heating samples to loosen molecular structures and enhancing access, binding, and detection with thermophilic CRISPR protein species.

Upon contact with a CRISPR system, the guide RNA interacts with the engineered CRISPR protein and a target sequence (which, as detailed above in section (II)(b), is adjacent to a PAM) in the nucleic acid and guides the engineered CRISPR protein to the target sequence, the engineered CRISPR protein undergoes structural changes upon accurate DNA target recognition by the guide RNA, and the engineered CRISPR protein forms a covalent bond with a 5′-phosphate within the target sequence thereby forming a CRISPR protein-nucleic acid complex (i.e., a CRISPR-tagged nucleic acid). The covalent linkage between the CRISPR protein and the nucleic acid is stable. For example, the covalent bond is insensitive to proteases and denaturing conditions (e.g., phenol, alkaline solutions, 8 M urea, SDS, heat, and the like).

(c) Detecting the CRISPR-Nucleic Acid Complex

The method further comprises detecting the CRISPR protein-nucleic acid complex or the CRISPR-tagged nucleic acid. The complex can be detected by a variety of detection means.

In embodiments in which the CRISPR system comprises at least one fluorescent marker domain or detectable label, the CRISPR-nucleic acid complex can be detected by fluorescence microscopy, such as e.g., confocal microscopy, multi photon microscopy, dynamic live cell imaging, FRET assay, FISH assay, and the like.

In other embodiments, the CRISPR-nucleic acid complex can be detected via an immunoassay such as immunohistochemistry, ELISA, Western blotting, or dot blotting. For example, the CRISPR-nucleic acid complex can be detected using antibodies against the CRISPR protein. In some embodiments, the detection signal can be amplified using a proximity ligation assay or modification thereof.

In yet other embodiments, the CRISPR-nucleic acid complex can be isolated from other nucleic acids and/or cellular macromolecules. Means for isolating chromosomal/genomic DNA are well known in the art, as are means for isolating cellular RNA. The CRISPR protein-nucleic acid complex can be detected via electrophoretic mobility shift (gel shift) assays. Alternatively, immunoprecipitation using antibodies against the CRISPR protein can be used to enrich for the CRISPR-nucleic acid complex. The immunoprecipitated CRISPR-nucleic acid complex can be subjected to DNA sequencing (e.g., nextgen sequencing). In other embodiments, the CRISPR-nucleic acid complex can also be isolated using biotin-avidin based systems.

(d) Applications

The methods disclosed herein can be used in a variety of therapeutic, diagnostic, industrial, and research applications.

In some embodiments, the methods can be used for specific and stable detection of nucleic acids of interest. The nucleic acids can be DNA or RNA. In some instances, the nucleic acids of interest can be visualized in situ.

In other embodiments, the methods can be used to determine the specificity of a CRISPR system by quantifying on-target and off-target events. For this, the covalent CRISPR-nucleic acid complexes can be immunoprecipitated and the associated nucleic acid can be sequenced. Moreover, the methods disclosed herein can be used for generalized unbiased CRISPR off-target discovery. Since the methods utilize covalent tagging and do not involve integration of an exogenous sequence such as in GUIDE-Seq (Tsai et al., Nature Biotechnology, 2015, 33:187-197), the methods disclosed herein may provide a more realistic quantitation of off-target action by CRISPR systems in live cells.

In other embodiments, the methods can be used to isolate fragments of interest from complex genomic DNA, and nextgen sequencing of the isolated fragments can be used to assemble and annotate genomic sequences.

In further embodiments, the methods disclosed herein can be used for diagnostic tests to establish the presence of a disease or disorder and/or for use in determining treatment options. Examples of suitable diagnostic tests include without limit detection of specific mutations in cancer cells (e.g., specific mutation in EGFR, HER2, and the like), detection of specific mutations associated with particular diseases (e.g., trinucleotide repeats, mutations in β-globin associated with sickle cell disease, specific SNPs, and the like), and detection of viruses (e.g., hepatitis C, Zika, and so forth).

In additional embodiments, the methods disclose herein can be used for gene regulation and modification. For example, covalent attachment of a CRISPR protein to genomic DNA could result in a useful genome modification, depending upon how the cell responds to the covalently attached CRISPR protein.

### Enumerated Embodiments

The following enumerated embodiments are presented to illustrate certain aspects of the present invention and are not intended to limit its scope.

1. A CRISPR protein engineered to comprise at least one modification such that the CRISPR protein is capable of forming a covalent bond with a 5′-phosphate within a target nucleic acid sequence.

2. The CRISPR protein of embodiment 1, wherein the at least one modification comprises a substitution of one or more amino acids, an insertion of one or more amino acids, a deletion of one or more amino acids, an insertion of a domain from a protein known to covalently bind nucleic acids, a replacement of a CRISPR protein domain with a domain from a protein known to covalently bind nucleic acids, or a combination thereof.

3. The CRISPR protein of embodiment 1 or 2, wherein the at least one modification comprises a substitution with a tyrosine residue, and/or the at least one modification comprises a substitution with a serine residue.

4. The CRISPR protein of embodiment 2, wherein the domain of the protein known to covalently bind nucleic acids is chosen from a topoisomerase, a recombinase, a rolling circle replication protein, an HUH endonuclease, an O⁶-alkylguanine-DNA alkyltransferase, or an acyl carrier protein.

5. The CRISPR protein of any one of embodiments 1 to 4, wherein the at least one modification is located within a nuclease domain of the CRISPR protein

6. The CRISPR protein of embodiment 5, wherein the nuclease domain is an HNH domain and the CRISPR protein is a Cas9 protein.

7. The CRISPR protein of embodiment 5, wherein the nuclease domain is a RuvC domain and the CRISPR protein is a Cas9 protein.

8. The CRISPR protein of embodiment 6 or 7, wherein the Cas9 protein comprises a catalytically inactive RuvC domain.

9. The CRISPR protein of embodiment 5, wherein the nuclease domain is a NUC domain and the CRISPR protein is a Cpf1 protein.

10. The CRISPR protein of embodiment 5, wherein the nuclease domain is a RuvC domain and the CRISPR protein is a Cpf1 protein.

11. The CRISPR protein of embodiment 9 or 10, wherein the Cpf1 protein comprises a catalytically inactive RuvC domain.

12. The CRISPR protein of any one of embodiments 1 to 11, further comprising at least one nuclear localization signal, at least one cell-penetrating domain, at least one marker domain, or combination thereof.

13. The CRISPR protein of any one of embodiments 1 to 12, further comprising at least one detectable label.

14. A nucleic acid encoding the CRISPR protein of any one of embodiments 1 to 12.

15. The nucleic acid of embodiment 14, wherein the nucleic acid is RNA or

DNA.

16. A system comprising the CRISPR protein of any one of embodiments 1 to 13 and a guide RNA.

17. The system of embodiment 16, wherein the guide RNA further comprises at least one detectable label.

18. The system of embodiment 16, wherein the guide RNA is a single molecule that is chemically synthesized or enzymatically synthesized, or the guide RNA comprises two molecules, which are chemically synthesized, enzymatically synthesized, or a combination thereof.

19. A nucleic acid encoding the system of embodiment 16.

20. The nucleic acid of embodiment 19, wherein sequence encoding the CRISPR protein and sequence encoding the guide RNA are each operably linked to a promoter control sequence.

21. The nucleic acid of embodiments 19 or 20, wherein the nucleic acid is a vector, and the vector is a plasmid vector, a viral vector, or a self-replicating viral RNA replicon.

22. A method for detecting a nucleic acid, the method comprising: (a) contacting the nucleic acid with a CRISPR system comprising (i) a CRISPR protein engineered to comprise at least one modification such that it is capable of forming a covalent linkage with a nucleic acid and (ii) a guide RNA, wherein the guide RNA guides the CRISPR protein to a target sequence in the nucleic acid and the CRISPR protein forms a covalent bond with a 5′-phosphate within the target sequence to form a CRISPR protein-nucleic acid complex; and (b) detecting the CRISPR protein-nucleic acid complex.

23. The method of embodiment 22, wherein the at least one modification comprises a substitution of one or more amino acids, an insertion of one or more amino acids, a deletion of one or more amino acids, an insertion of a domain from a protein known to covalently bind nucleic acids, a replacement of a CRISPR protein domain with a domain from a protein known to covalently bind nucleic acids, or a combination thereof.

24. The method of embodiment 22 or 23, wherein the at least one modification comprises a substitution with a tyrosine residue, and/or the at least one modification comprises a substitution with a serine residue.

25. The method of embodiment 22, wherein the domain of the protein known to covalently bind nucleic acids is chosen from a topoisomerase, a recombinase, a rolling circle replication protein, an HUH endonuclease, an 0⁶-alkylguanine-DNA alkyltransferase, or an acyl carrier protein.

26. The method of any one of embodiments 22 to 25, wherein the at least one modification is located within a nuclease domain of the CRISPR protein.

27. The method of embodiment 26, wherein the nuclease domain is an HNH domain or a RuvC domain and the CRISPR protein is a Cas9 protein.

28. The method of embodiment 27, wherein the Cas9 protein comprises a catalytically inactive RuvC domain.

29. The method of embodiment 26, wherein the nuclease domain is a NUC domain or a RuvC domain and the CRISPR protein is a Cpf1 protein.

30. The method of embodiment 29, wherein the Cpf1 protein comprises a catalytically inactive RuvC domain.

31. The method of any one of embodiments 22 to 30, wherein the CRISPR protein further comprises at least one nuclear localization signal, at least one cell-penetrating domain, at least one marker domain, at least one detectable label, or combination thereof, and/or the guide RNA comprises at least one detectable label.

32. The method of any one of embodiments 22 to 31, wherein the nucleic acid that is detected is DNA or RNA.

33. The method of any one of embodiments 22 to 32, wherein the nucleic acid is within a cell, and the cell is in vivo or ex vivo, or the cell is a cultured cell or a cell line cell.

34. The method of embodiment 33, wherein the cell is a live cell or a fixed cell.

35. The method of embodiment 33 or 34, wherein the cell is a eukaryotic cell.

36. The method of any one of embodiments 33 to 35, wherein the cell is live and the contacting step comprises introducing into the cell a complex comprising the CRISPR protein and the guide RNA, the CRISPR protein and a nucleic acid encoding the guide RNA, or a nucleic acid encoding the CRISPR protein and a nucleic acid encoding the guide RNA.

37. The method of embodiment 36, wherein the nucleic acid encoding the CRISPR protein is RNA.

38. The method of embodiment 36, wherein the nucleic acid encoding the CRISPR protein is DNA and the nucleic acid encoding the guide RNA is DNA.

39. The method of embodiment 38, wherein the nucleic acids are part of a vector.

40. The method of any one of embodiments 33 to 35, wherein the cell is fixed and the contacting step comprises introducing into the cell a complex comprising the CRISPR protein and the guide RNA.

41. The method of any one of embodiments 22 to 32, wherein nucleic acid is in vitro.

42. The method of any one of embodiments 22 to 41, wherein the detecting comprises fluorescence microscopy, immunohistochemistry, ELISA, immunoprecipitation, and/or DNA sequencing.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x±5%.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudouridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the disclosure.

### Example 1. Rational Design of Covalent CRISPR Tags

CRISPR proteins having the ability to form covalent attachments with target DNA can be generated using a rational design approach based on existing protein structural information. Determination of the spatial location of the tyrosine or serine residues required to establish a covalent bond can be advised by existing tertiary structural information on CRISPR complexes. For example, the three dimensional structure of Cas9 and Cpf1 systems has been determined and, more importantly, the molecular dynamics of protein structure in relation to target DNA binding have been elucidated in enough detail (Chen et al., Nature, 2017, 550:407-410; Gao et al., Cell Research, 2016, 26(8):901-913) to advise placement of Tyr or Ser residues within the mobile HNH nuclease domain (Cas9) or the mobile NUC domain (Cpf1). Most importantly, placement of Tyr or Ser residues within these domains would serve to make covalent attachment highly dependent on accurate DNA target recognition by the guide RNA as communicated structurally to the HNH or NUC domains via REC or equivalent domains in contact with the guide RNA-target DNA duplex.

A favorable composition of mutations for a stable covalent attachment comprises: (1) Maintenance of existing HNH (Cas9) or NUC (Cpf1) domain to retain REC domain-induced rotational specificity upon guide RNA target binding. (2) Placement of a Tyr or Ser residue within the HNH or NUC domain, and in spatial proximity to the point of DNA cleavage by the critical nuclease residues of wild-type Cas9 or Cpf1 nucleases. In addition to changes to the Cas9 or Cpf1 nuclease residues critical for breakage of the phosphate backbone, additional spatially adjacent residues maybe be altered to assist with the required catalytic process for breakage and covalent attachment to the phosphate backbone of the DNA target and these choices can be advised by review of the existing literature. For example, the critical tyrosine residue for covalent attachment in RepA protein of rolling circle plasmid pC194 is suspected, based on current biochemical data (Noirot-Gros F. et al. The EMBO Journal, 1994, 13(18):4412-4420), to receive catalytic assistance from a spatially adjacent glutamate residue interacting with the phosphate backbone via a magnesium cation. The phage phiX174 genome uses a process similar to plasmid pC194, however the Rep protein uses two tyrosine residues to achieve covalent binding (Noirot-Gros et al., 1994). (3) Optional (perhaps in live cells and when mutations are focused on the HNH and NUC regions) inactivation of the RuvC endonuclease active site to keep the second DNA target strand intact, thereby not activating additional host cellular DNA repair responses that may modify or destroy the covalent CRISPR-DNA complex.

In addition to the more subtle replacement of individual amino acids within CRISPR proteins, entire HNH, NUC, or RuvC domains can be swapped with domains from proteins known for covalent attachment to DNA (e.g., derived from those listed above in paragraph [0022] or similar proteins in the extended literature). Favorable choices for complete or partial replacement of HNH, NUC, or RuvC domains would be those of similar molecular weight and shape as the replaced HNH, NUC, or RuvC domain. Even more favorable choices for complete or partial replacement of the HNH, NUC, and RuvC domains would be heterologous domains with similar molecular weight, shape, and placement of amino acid residues in relation to the target DNA strand. Since many proteins known to covalently bind DNA also operate on single stranded DNA substrates, the single stranded DNA environment known to be created within the Cas9 and Cpf1 protein DNA target-bound structures creates a favorable environment to test domain swapping or grafting of alternate amino acids to achieve covalent attachment.

While the HNH and NUC domains of Cas9 and Cpf1 are a favorable choice for tyrosine, serine, and other substitutions, in some cases these domains may remain unchanged with tyrosine, serine or other substitutions focused on the RuvC domain. Furthermore, the HNH or NUC domains may contain substitutions known to inactivate their nuclease activity, keeping the gRNA-bound target DNA strand intact while the RuvC domain uses amino acid substitutions to achieve a covalent bond with the complementary target DNA strand.

The extent and stability of covalent bonding between the modified CRISPR protein and the target DNA can be assessed using standard procedures. For example, fluorescent or radiolabeled target DNA substrates can be used to detect and quantify CRISPR protein:DNA binding upon immunoprecipitation of the CRISPR protein, or exposure of the complex to materials which differentially bind protein vs. DNA (e.g., nitrocellulose membranes). Bound complexes suspected to contain covalent bonds between target DNAs and engineered CRISPR proteins can be washed with harsh reagents known to denature or degrade proteins (i.e., urea, guanidine, SDS, proteinase K, etc.) to assess the stability and nature of the association.

### Example 2. Semi-Rational Design of Covalent CRISPR Tags

A semi-rational approach can be employed in which tyrosine or serine residues are placed at many semi-randomly selected locations within the HNH, NUC, and/or RuvC domains followed by a medium-throughput screen (e.g., gel shift, immunoprecipitation, etc.) to assess the extent and stability of covalent bonding. Alternatively, a large collection of semi-rational designs could be screened using the scheme outlined in Example 3 for isolation and sequencing of plasmid-protein covalent complexes. This semi-rational approach could include: (1) error-prone PCR, (2) selected randomization via in silico design oligo synthesis and production of modified synthetic DNA fragments, (3) homology-based DNA shuffling methods such as those dependent upon incomplete PCR or DNaseI fragmentation and re-amplification.

### Example 3. Irrational Design of Covalent CRISPR Tags

An irrational approach based on efficient molecular selection can be devised which evolves mutants which achieve efficient and stable covalent attachment. The entire amino acid sequence of Cas9 (or Cpf1) can be randomly mutated with single substitutions of tyrosine, serine, or other single and combinatorial formats (DNA shuffling, etc.) to determine the optimal placement of amino acid substitutions. One format for an efficient molecular screen would be to place a CRISPR target site within an E. coli plasmid which also expresses the mutant CRISPR protein. Upon expression of the CRISPR protein and a guide RNA which directs it to a plasmid-harbored DNA target site, the CRISPR protein would bind to the target site and covalently attach itself to the plasmid from which it was expressed (or to another identical plasmid within the same E. coli cell). Upon cell lysis, the CRISPR protein can be isolated using a tag (e.g., biotin, poly-histidine, FLAG, or other). Following extensive washing, a fraction of the covalently attached target sites would also contain the plasmid-harbored genetic information encoding the modified CRISPR protein. The nucleic acid information encoding the modified CRISPR protein mutant which successfully and stably formed a covalent bond with its target could then be amplified and/or sequenced, then subcloned for further confirmation of activity, use, or subsequent rounds of molecular evolution.

What is claimed is:

1. A CRISPR protein engineered to comprise at least one modification such that the CRISPR protein is capable of forming a covalent bond with a 5′-phosphate within a target nucleic acid sequence.

2. The CRISPR protein of claim 1, wherein the at least one modification comprises a substitution of one or more amino acids, an insertion of one or more amino acids, a deletion of one or more amino acids, an insertion of a domain from a protein known to covalently bind nucleic acids, a replacement of a CRISPR protein domain with a domain from a protein known to covalently bind nucleic acids, or a combination thereof.

3. The CRISPR protein of claim 1, wherein the at least one modification comprises a substitution with a tyrosine residue, and/or the at least one modification comprises a substitution with a serine residue.

4. The CRISPR protein of claim 2, wherein the domain of the protein known to covalently bind nucleic acids is chosen from a topoisomerase, a recombinase, a rolling circle replication protein, an HUH endonuclease, an 0⁶-alkylguanine-DNA alkyltransferase, or an acyl carrier protein.

5. The CRISPR protein of claim 1, wherein the at least one modification is located within a nuclease domain of the CRISPR protein.

6. The CRISPR protein of claim 5, wherein the nuclease domain is an HNH domain or a RuvC domain and the CRISPR protein is a Cas9 protein.

7. The CRISPR protein of claim 6, wherein the Cas9 protein comprises a catalytically inactive RuvC domain.

8. The CRISPR protein of claim 5, wherein the nuclease domain is a NUC domain or a RuvC domain and the CRISPR protein is a Cpf1 protein.

9. The CRISPR protein of claim 8, wherein the Cpf1 protein comprises a catalytically inactive RuvC domain.

10. The CRISPR protein of claim 1, further comprising at least one nuclear localization signal, at least one cell-penetrating domain, at least one marker domain, or combination thereof.

11. The CRISPR protein of claim 1, further comprising at least one detectable label.

12. A nucleic acid encoding the CRISPR protein of claim 1.

13. The nucleic acid of claim 12, wherein the nucleic acid is RNA or DNA.

14. A system comprising the CRISPR protein of claim 1 and a guide RNA.

15. The system of claim 14, wherein the guide RNA further comprises at least one detectable label.

16. The system of claim 15, wherein the guide RNA is a single molecule that is chemically synthesized or enzymatically synthesized, or the guide RNA comprises two molecules, which are chemically synthesized, enzymatically synthesized, or a combination thereof.

17. A nucleic acid encoding the system of claim 14.

18. The nucleic acid of claim 17, wherein sequence encoding the CRISPR protein and sequence encoding the guide RNA are each operably linked to a promoter control sequence.

19. The nucleic acid of claim 18, wherein the nucleic acid is a vector, and the vector is a plasmid vector, a viral vector, or a self-replicating viral RNA replicon.

20. A method for detecting a nucleic acid, the method comprising: (a) contacting the nucleic acid with a CRISPR system comprising (i) the CRISPR protein of claim 1, and (ii) a guide RNA, wherein the guide RNA guides the CRISPR protein to a target sequence in the nucleic acid and the CRISPR protein forms a covalent bond with a 5′-phosphate within the target sequence to form a CRISPR protein-nucleic acid complex; and (b) detecting the CRISPR protein-nucleic acid complex.


SEQUENCE LISTING (30 sequences)

SEQ ID NO: 1 - protein, 7 aa, Unknown
  Description of Unknown nuclear localization signal sequence
PKKKRKV

SEQ ID NO: 2 - protein, 7 aa, Unknown
  Description of Unknown nuclear localization signal sequence
PKKKRRV

SEQ ID NO: 3 - protein, 16 aa, Unknown
  Description of Unknown nuclear localization signal sequence
KRPAATKKAGQAKKKK

SEQ ID NO: 4 - protein, 11 aa, Unknown
  Description of Unknown nuclear localization signal sequence
YGRKKRRQRRR

SEQ ID NO: 5 - protein, 9 aa, Unknown
  Description of Unknown nuclear localization signal sequence
RKKRRQRRR

SEQ ID NO: 6 - protein, 9 aa, Unknown
  Description of Unknown nuclear localization signal sequence
PAAKRVKLD

SEQ ID NO: 7 - protein, 11 aa, Unknown
  Description of Unknown nuclear localization signal sequence
RQRRNELKRSP

SEQ ID NO: 8 - protein, 8 aa, Unknown
  Description of Unknown nuclear localization signal sequence
VSRKRPRP

SEQ ID NO: 9 - protein, 8 aa, Unknown
  Description of Unknown nuclear localization signal sequence
PPKKARED

SEQ ID NO: 10 - protein, 8 aa, Unknown
  Description of Unknown nuclear localization signal sequence
PQPKKKPL

SEQ ID NO: 11 - protein, 12 aa, Unknown
  Description of Unknown nuclear localization signal sequence
SALIKKKKKMAP

SEQ ID NO: 12 - protein, 7 aa, Unknown
  Description of Unknown nuclear localization signal sequence
PKQKKRK

SEQ ID NO: 13 - protein, 10 aa, Unknown
  Description of Unknown nuclear localization signal sequence
RKLKKKIKKL

SEQ ID NO: 14 - protein, 10 aa, Unknown
  Description of Unknown nuclear localization signal sequence
REKKKFLKRR

SEQ ID NO: 15 - protein, 20 aa, Unknown
  Description of Unknown nuclear localization signal sequence
KRKGDEVDGVDEVAKKKSKK

SEQ ID NO: 16 - protein, 17 aa, Unknown
  Description of Unknown nuclear localization signal sequence
RKCLQAGMNLEARKTKK

SEQ ID NO: 17 - protein, 38 aa, Unknown
  Description of Unknown nuclear localization signal sequence
NQSSNFGPMKGGNFGGRSSGPYGGGGQYFAKPRNQGGY

SEQ ID NO: 18 - protein, 42 aa, Unknown
  Description of Unknown nuclear localization signal sequence
RMRIZFKNKGKDTAELRRRRVEVSVELRKAKKDEQILKRRNV

SEQ ID NO: 19 - protein, 20 aa, Unknown
  Description of Unknown cell-penetrating domain sequence
GRKKRRQRRRPPQPKKKRKV

SEQ ID NO: 20 - protein, 19 aa, Unknown
  Description of Unknown cell-penetrating domain sequence
PLSSIFSRIGDPPKKKRKV

SEQ ID NO: 21 - protein, 24 aa, Unknown
  Description of Unknown cell-penetrating domain sequence
GALFLGWLGAAGSTMGAPKKKRKV

SEQ ID NO: 22 - protein, 27 aa, Unknown
  Description of Unknown cell-penetrating domain sequence
GALFLGFLGAAGSTMGAWSQPKKKRKV

SEQ ID NO: 23 - protein, 21 aa, Unknown
  Description of Unknown cell-penetrating domain sequence
KETWWETWWTEWSQPKKKRKV

SEQ ID NO: 24 - protein, 11 aa, Unknown
  Description of Unknown cell-penetrating domain sequence
YARAAARQARA

SEQ ID NO: 25 - protein, 11 aa, Unknown
  Description of Unknown cell-penetrating domain sequence
THRLPRRRRRR

SEQ ID NO: 26 - protein, 11 aa, Unknown
  Description of Unknown cell-penetrating domain sequence
GGRRARRRRRR

SEQ ID NO: 27 - protein, 12 aa, Unknown
  Description of Unknown cell-penetrating domain sequence
RRQRRTSKLMKR

SEQ ID NO: 28 - protein, 27 aa, Unknown
  Description of Unknown cell-penetrating domain sequence
GWTLNSAGYLLGKINLKALAALAKKIL

SEQ ID NO: 29 - protein, 33 aa, Unknown
  Description of Unknown cell-penetrating domain sequence
KALAWEAKLAKALAKALAKHLAKALAKALKCEA

SEQ ID NO: 30 - protein, 16 aa, Unknown
  Description of Unknown cell-penetrating domain sequence
RQIKIWFQNRRMKWKK